(19) United States
(12) Patent Application Publication
(10) Pub. No.: US 2020/0074628 A1
(43) Pub. Date: Mar. 5, 2020

(54) IMAGE PROCESSING APPARATUS, IMAGING SYSTEM, IMAGE PROCESSING METHOD AND COMPUTER READABLE RECODING MEDIUM

(71) Applicant: OLYMPUS CORPORATION, Tokyo (JP)

(72) Inventors: Takeshi OTSUKA, Tokyo (JP); Chika IZUMI, Tokyo (JP)

(73) Assignee: OLYMPUS CORPORATION, Tokyo (JP)

(21) Appl. No.: 16/663,435

(22) Filed: Oct. 25, 2019

Related U.S. Application Data
(63) Continuation of application No. PCT/JP2017/016635, filed on Apr. 26, 2017.

(51) Int. Cl. G06T 7/00; G01N 33/483

(52) U.S. Cl. CPC G06T 7/0012; G06T 2207/10024; G06T 2207/30024; G01N 33/483

(57) ABSTRACT

An image processing apparatus processes a pathological specimen image obtained by imaging a pathological specimen, and includes a processor including hardware configured to: perform machine learning independently on a plurality of training images for machine learning that are prepared based on a plurality of different standards, respectively; apply each of a plurality of results of machine learning to all the training images, respectively; learn by machine learning a diagnosis ambiguous area whose corresponding result of diagnosis is ambiguous based on an area on which different determinations are made between at least two of the results of application; extract the diagnosis ambiguous area in the pathological specimen image based on a result of the machine learning performed by the diagnosis ambiguous area learning unit; and generate a diagnosis image that enables the diagnosis ambiguous area that is extracted by the diagnosis ambiguous area extractor to be distinguished from other areas.

This application is a continuation of International Application No. PCT/JP2017/016635, filed on Apr. 26, 2017, the entire contents of which are incorporated herein by reference.

### BACKGROUND

The present disclosure relates to an image processing apparatus, an imaging system, an image processing method and a computer readable recording medium.

In pathological diagnosis, a sample is excised from a patient and a pathological specimen is prepared from the sample, and then the pathological specimen is observed with a microscope and a diagnosis is made on whether there is a disease or on the degree of disease from the tissue form or the staining condition. The pathological specimen is prepared by performing steps of cutting, fixing, embedding, slicing, staining, and sealing on the excised sample. Particularly, a method of applying transmission light to the pathological specimen and performing magnification observation has been employed from long ago.

In pathological diagnosis, a primary diagnosis is made and, when a disease is suspected, a secondary diagnosis is made.

In the primary diagnosis, a diagnosis is made on whether there is a disease from the tissue form of the pathological specimen. For example, Hematoxylin-Eosin staining (HE staining) is performed on the specimen so that cell nuclei, bone tissue, etc., are stained in bluish purple and cell cytoplasm, connective tissue, electrolyte, etc., are stained in red. A pathologist diagnose make a diagnosis on whether there if a disease morphologically from the tissue form.

In the secondary diagnosis, a diagnosis is made on whether there is disease from expression of molecules. For example, immunostaining is performed on the specimen to visualize expression of molecules from antigen-antibody reaction. The pathologist makes a diagnosis on whether there is a disease from expression of molecules. The pathologist selects an appropriate treatment method from a positive rate (ratio of negative cells and positive cells).

A camera is connected to the microscope to image the pathological specimen so that images of the pathological specimen are captured. In a virtual microscopic system, images of the whole pathological specimen are captured. Images of the pathological specimen will be referred to as pathological specimen images below. Such pathological specimen images are used in various scenes from education to remote pathology.

In recent years, methods of digitally supporting diagnosis from pathological specimen images (digital diagnostic support below) have been developed. Digital diagnostic support includes methods of imitating a diagnosis made by a pathologist by classical image processing and a machine learning method using a large volume of training data (training images). For machine learning, linear determination, deep learning, etc., are used.

In counting of molecule expression in the secondary diagnosis, it is possible to imitate a diagnosis made by a pathologist and the imitation can be also realized by the classical image processing method. On the other hand, in morphological analysis in the primary diagnosis, it is difficult to imitate a diagnosis made by a pathologist and a method of machine learning performed on a large volume of data is used.

Current pathological diagnosis increases the work of pathologists due to a shortage of pathologists and it is expected to reduce the work by digital diagnostic support.

Various variations occur in pathological specimen. When a pathological specimen out of the standard is used, an appropriate diagnosis cannot be made.

For example, the variations occur in a specimen preparation condition, such as the depth of color, depending on the preference of the pathologist, the skill of a clinical laboratory technician, and the performance of a specimen preparation facility. When a pathological specimen whose corresponding specimen preparation condition is out of the standards is used, an appropriate diagnosis cannot be made. Thus, a technique to determine whether the staining condition of the pathological specimen is adequate in advance has been proposed (see Japanese Laid-open Patent Publication No. 2008-185337). When the staining condition is at the standards, the technique described in Japanese Laid-open Patent Publication No. 2008-185337 gives an analysis and, when the staining condition is not at the standards, gives no analysis or performs staining again or performs digital correction to the standards.

Furthermore, for example, even in the same specimen, positivity differs depending on the field of view. The treatment method differs depending on the positivity. Fields of view with different positivities correspond to different treatment methods and therefore it is necessary to choose fields of view appropriately. To deal with this, a technique to represent only areas that meet a necessary positivity rate in staining a subject as areas to be diagnosed has been proposed (for example, refer to Japanese Laid-open Patent Publication No. 2015-38467).

### SUMMARY

According to one aspect of the present disclosure, there is provided an image processing apparatus for processing a pathological specimen image obtained by imaging a pathological specimen, the image processing apparatus including a processor including hardware, the processor being configured to: perform machine learning independently on a plurality of training images for machine learning that are prepared based on a plurality of different standards, respectively; apply each of a plurality of results of machine learning to all the training images, respectively; learn by machine learning a diagnosis ambiguous area whose corresponding result of diagnosis is ambiguous based on an area on which different determinations are made between at least two of the results of application; extract the diagnosis ambiguous area in the pathological specimen image based on a result of the machine learning performed by the diagnosis ambiguous area learning unit; and generate a diagnosis image that enables the diagnosis ambiguous area that is extracted by the diagnosis ambiguous area extractor to be distinguished from other areas.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a block diagram illustrating a configuration of an imaging system according to a first embodiment;

FIG. 2 is a diagram schematically illustrating a configuration of the imaging device illustrated in FIG. 1;

FIG. 3 is a diagram illustrating exemplary spectral sensitivity characteristics of the RGB camera illustrated in FIG. 2;

FIG. 4 is a diagram illustrating exemplary spectral characteristics of the first filter illustrated in FIG. 2;

FIG. 5 is a diagram illustrating exemplary spectral characteristics of the second filter illustrated in FIG. 2;

FIG. 6 is a flowchart illustrating a machine learning method for a diagnosis ambiguous area;

FIG. 7 is a diagram illustrating a training image;

FIG. 8 is a diagram illustrating a training image;

FIG. 9 is a diagram illustrating step S2 represented in FIG. 6;

FIG. 10 is a flowchart illustrating a method processing a pathological specimen image;

FIG. 11 is a diagram illustrating an exemplary diagnosis image;

FIG. 12 is a diagram illustrating an exemplary diagnosis image;

FIG. 13 is a diagram illustrating an exemplary diagnosis image;

FIG. 14 is a diagram illustrating an exemplary diagnosis image;

FIG. 15 is a diagram illustrating an exemplary diagnosis image;

FIG. 16 is a flowchart illustrating a machine learning method for a diagnosis ambiguous area according to a second embodiment;

FIG. 17 is a diagram illustrating the machine learning method for a diagnosis ambiguous area illustrated in FIG. 16;

FIG. 18 is a flowchart illustrating a machine learning method for a diagnosis ambiguous area according to a third embodiment;

FIG. 19 is a diagram illustrating the machine learning method for a diagnosis ambiguous area illustrated in FIG. 18;

FIG. 20 is a flowchart illustrating a machine learning method for a diagnosis ambiguous area according to a fourth embodiment;

FIG. 21 is a diagram illustrating the machine learning method for a diagnosis ambiguous area illustrated in FIG. 20;

FIG. 22 is a diagram illustrating a modification of the first to fourth embodiments;

FIG. 23 is a diagram illustrating the modification of the first to fourth embodiments; and

FIG. 24 is a diagram illustrating the modification of the first to fourth embodiments.

### DETAILED DESCRIPTION

Modes for carrying out the present disclosure (“embodiments” below) will be described below with reference to the drawings. The embodiments described below do not limit the disclosure. Furthermore, like parts are denoted with like reference numerals in the drawings.

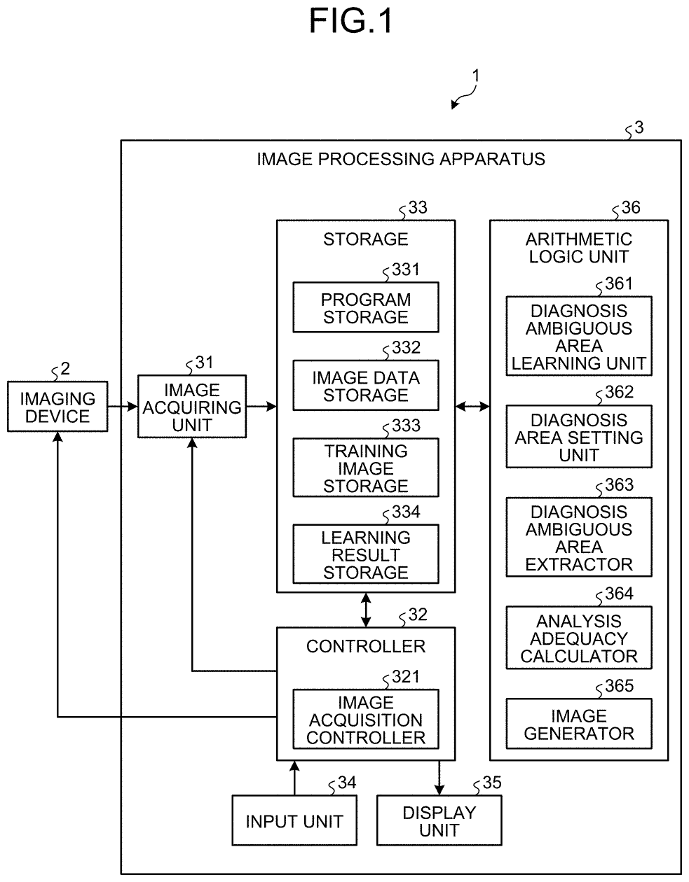

FIG. 1 is a block diagram illustrating a configuration of an imaging system 1 according to a first embodiment.

The imaging system 1 is a system that images a pathological specimen on which staining has been performed and processes a pathological specimen image obtained by the imaging.

For the staining performed on the pathological specimen, cell nuclei immunostaining using Ki-67, ER, PgR, or the like, as an antibody; cell membrane immunostaining using HER2, or the like, as an antibody; cytoplasmic immunostaining using serotonin, or the like, as an antibody; cell nuclei counterstaining using hematoxylin (H) as a pigment; and cytoplasmic counterstaining using eosin (E) as a pigment can be exemplified.

As illustrated in FIG. 1, the imaging system 1 includes an imaging device 2 and an image processing apparatus 3.

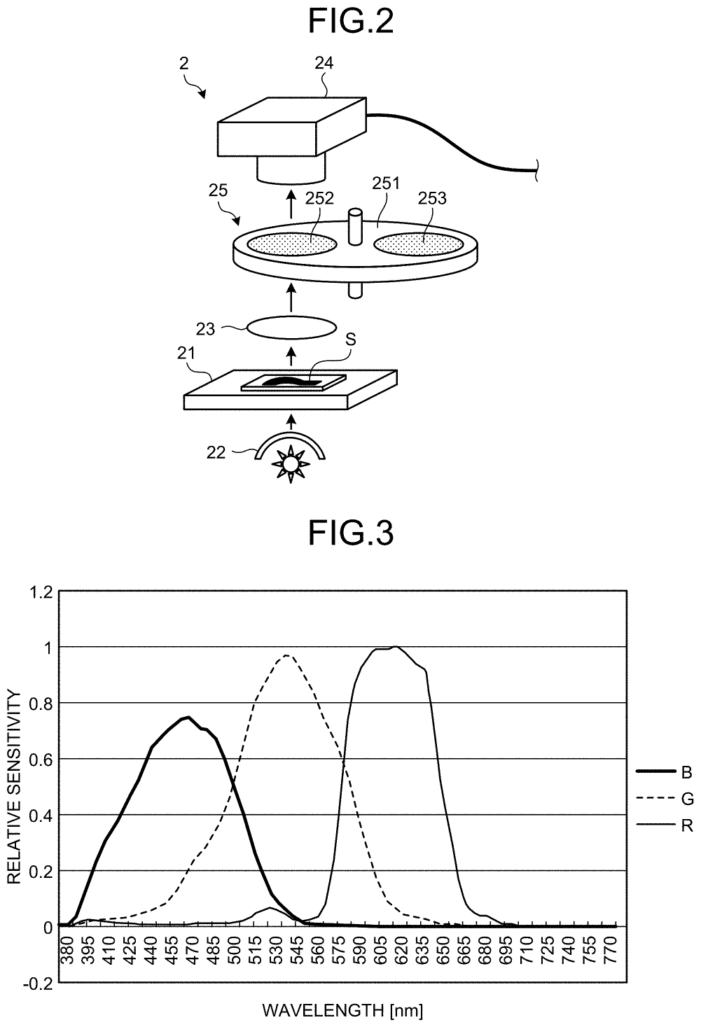

FIG. 2 is a diagram schematically illustrating a configuration of the imaging device 2.

The imaging device 2 is a device that acquires a pathological specimen image of a pathological specimen S (FIG. 2). In the first embodiment, the imaging device 2 is configured as a device that acquires a pathological specimen image that is a multiband image. As illustrated in FIG. 2, the imaging device 2 includes a stage 21, an illuminator 22, an image forming optical system 23, an RGB camera 24, and a filter unit 25.

The stage 21 is a part on which the pathological specimen S is placed and is configured to, under the control of the image processing apparatus 3, move such that an area to be observed in the pathological specimen S can be changed.

Under the control of the image processing apparatus 3, the illuminator 22 applies illumination light to the pathological specimen S that is placed on the stage 21.

The image forming optical system 23 forms, on the RGB camera 24, an image of the transmitted light that is applied to the pathological specimen S and that is transmitted through the pathological specimen S.

FIG. 3 is a diagram illustrating exemplary spectral sensitivity characteristics of the RGB camera 24.

The RGB camera 24 is a part corresponding to an imaging unit according to the disclosure and includes imaging elements, such as a charge coupled device (CCD) or a complementary metal oxide semiconductor (CMOS). Unser the control of the image processing apparatus 3, the RGB camera 24 captures an image of the transmitted light having been transmitted through the pathological specimen S. The RGB camera 24 has, for example, spectral sensitivity characteristics of each of bands of red (R), green (G) and blue (B) represented in FIG. 3.

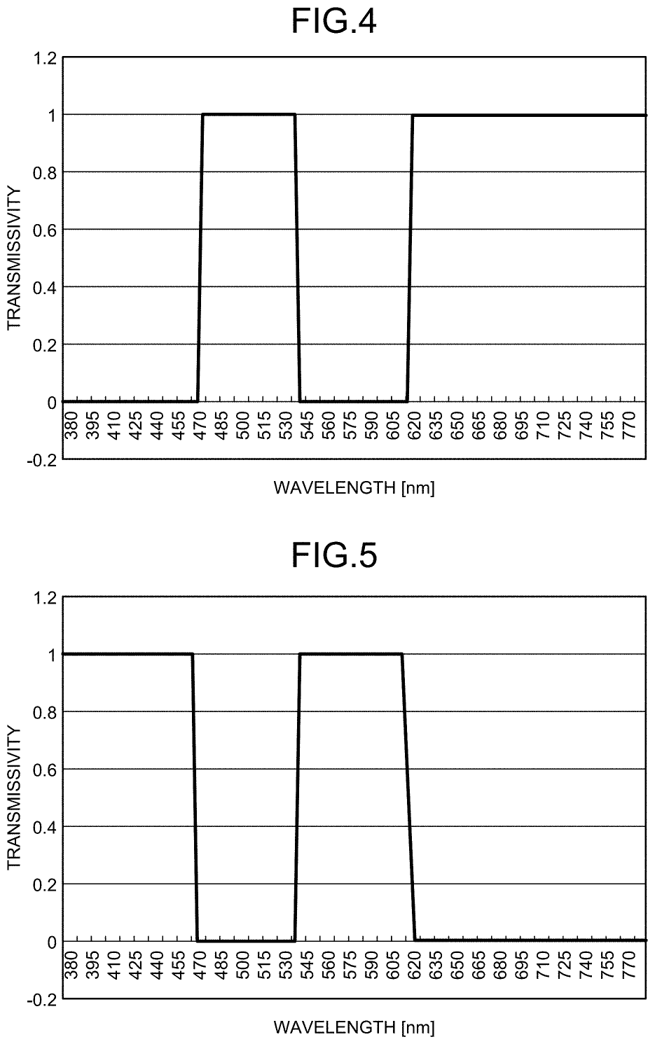

FIG. 4 is a diagram illustrating exemplary spectral characteristics of a first filter 252. FIG. 5 is a diagram illustrating exemplary spectral characteristics of the first filter 252.

The filter unit 25 is provided on an optical path from the image forming optical system 23 to the RGB camera 24 and limits the wavelength band of the light of which image is formed on the RGB camera 24 to a given range. As illustrated in FIG. 2, the filter unit 25 includes a filter wheel 251 that is rotatable under the control of the image processing apparatus 3; and first and second filters 252 and 253 having different spectral sensitivity characteristics (the spectral characteristics in FIGS. 4 and 5) such that each of the RGB transmission wavelength bands is divided into two.

Under the control of the image processing apparatus 3, as described below, the imaging device 2 acquires a pathological specimen image (multiband image) of the pathological specimen S.

First of all, the imaging device 2 positions the first filter 252 on the optical path from the illuminator 22 to the RGB camera 24 and applies illumination light from the illuminator 22 to the pathological specimen S. The RGB camera 24 images the transmitted light that is transmitted through the pathological specimen S and then through the first filter 252 and the image forming optical system 23 (first imaging).

The imaging device 2 then positions the second filter 253 on the optical path from the illuminator 22 to the RGB camera 24 and performs second imaging as in the first imaging.

Accordingly, each of the first imaging and the second imaging captures images of three bands different from one another so that pathological specimen images of six bands are acquired in total.

The number of filters that are provided in the filter unit 25 is not limited to two. Three or more filters may be provided to acquire more band images. The imaging device 2 may be configured to acquire only RGB images with the RGB camera 24 without the filter unit 25. Instead of the filter unit 25, a liquid crystal tunable filter or an acoustic optical tunable filter whose spectral characteristic is changeable may be used. A pathological specimen image (multiband image) may be acquired by switching between a plurality of lights whose spectral characteristics are different and applying the lights. The imaging unit of the disclosure is not limited to the RGB camera 24 and a monochrome camera may be used.

The image processing apparatus 3 is an apparatus that processes a pathological specimen image of the pathological specimen S that is acquired by the imaging device 2. As illustrated in FIG. 1, the image processing apparatus 3 includes an image acquiring unit 31, a controller 32, a storage 33, an input unit 34, a display unit 35, and an arithmetic logic unit 36.

The image acquiring unit 31 is configured as appropriate according to the mode of the imaging system 1. For example, when the imaging device 2 is connected to the image processing apparatus 3, the image acquiring unit 31 is formed of an interface that loads the pathological specimen image (image data) that is output from the imaging device 2. When a server for saving pathological specimen images that are acquired by the imaging device 2 is set, the image acquiring unit 31 is formed of a communication device, or the like, that is connected to the server and communicates data with the server to acquire a pathological specimen image. Alternatively, the image acquiring unit 31 may be formed of a reader device to which a portable recording medium is detachably attached and that reads a pathological specimen image that is recorded in the recording medium.

The controller 32 is formed using a central processing unit (CPU), or the like. The controller 32 includes an image acquisition controller 321 that controls operations of the image acquiring unit 31 and the imaging device 2 and acquires a pathological specimen image. The controller 32 controls operations of the image acquiring unit 31, the imaging device 2 and the display unit 35 based on an input signal that is input from the input unit 34, the pathological specimen image that is input from the image acquiring unit 31, and programs and data that are stored in the storage 33.

The storage 33 is formed of an information storage device, such as various IC memories including a read only memory (ROM) like an updatable and recordable flash memory or a random access memory (RAM), a hard disk that is incorporated or that is connected with a data communication terminal, or a CD-ROM, and an information write-read device for information to be read from or to be written in the information storage device. The storage 33 includes a program storage 331, an image data storage 332, a training image storage 333, and a learning result storage 334.

The program storage 331 stores an image processing program that is executed by the controller 32.

The image data storage 332 stores the pathological specimen image that is acquired by the image acquiring unit 31.

The training image storage 333 stores a training image for machine learning at the arithmetic logic unit 36.

The learning result storage 334 stores the result of machine learning at the arithmetic logic unit 36.

The input unit 34 is formed of, for example, various input devices including a keyboard, a mouse, a touch panel, and various switches, and outputs input signals corresponding to operation inputs to the controller 32.

The display unit 35 is achieved with a display device, such as a liquid crystal display (LCD) or an electro luminescence (EL) display, or a cathode ray tube (CRT) display, and the display unit 35 displays various screens based on a display signal that is input from the controller 32.

The arithmetic logic unit 36 is formed using a CPU, etc. As illustrated in FIG. 1, the arithmetic logic unit 36 includes a diagnosis ambiguous area learning unit 361, a diagnosis area setting unit 362, a diagnosis ambiguous area extractor 363, an analysis adequacy calculator 364, and an image generator 365.

The diagnosis ambiguous area learning unit 361 reads a training image that is stored in the training image storage 333 and, based on the training image, learns by machine learning a diagnosis ambiguous area whose corresponding diagnostic result is ambiguous (for example, an area whose corresponding diagnostic result differs among a plurality of medical facilities, a plurality of pathologists, or in a single pathologist). Linear determination and deep learning can be exemplified as the machine learning. The diagnosis ambiguous area learning unit 361 stores the results of machine learning in the learning result storage 334.

The diagnosis area setting unit 362 sets a diagnosis subject area to be diagnosed in the pathological specimen image (the pathological specimen image displayed on the display unit 35).

The diagnosis ambiguous area extractor 363 reads the pathological specimen image that is stored in the image data storage 332 and, based on the machine learning result that is stored in the learning result storage 334, extracts a diagnosis ambiguous area in the pathological specimen image.

The analysis adequacy calculator 364 calculates an analysis adequacy of the diagnosis subject area in the pathological specimen image based on the diagnosis ambiguous areas that are extracted by the diagnosis ambiguous area extractor 363. A ratio of the diagnosis ambiguous areas in the diagnosis subject area with respect to the whole diagnosis subject area can be exemplified as the analysis adequacy. In other words, a higher the analysis adequacy represents that the diagnosis subject area is not inadequate for diagnosis.

The image generator 365 generates a diagnosis image for diagnosis that enables the diagnosis ambiguous area in the pathological specimen image to be distinguished from other areas and that corresponds to the analysis adequacy that is calculated by the analysis adequacy calculator 364.

Operations of the image processing apparatus 3 will be described.

A diagnosis ambiguous area machine learning method and a pathological specimen image processing method will be described sequentially below as operations of the image processing apparatus 3 (an image processing method according to the disclosure).

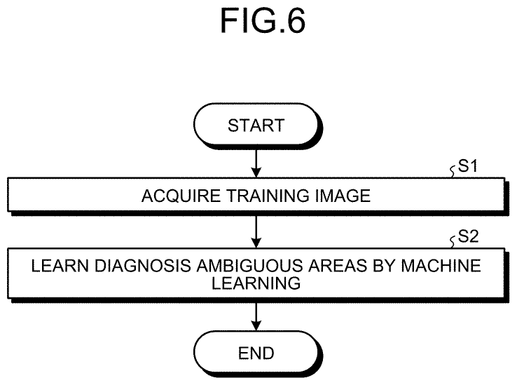

FIG. 6 is a flowchart illustrating the diagnosis ambiguous area machine learning method.

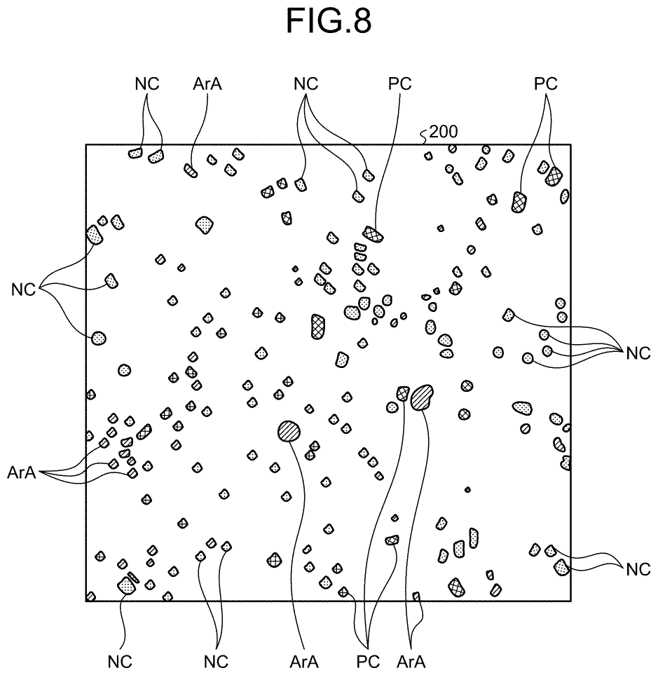

First of all, the diagnosis ambiguous area learning unit 361 reads a training image 200 that is stored in the training image storage 333 (refer to FIG. 8) (step S1).

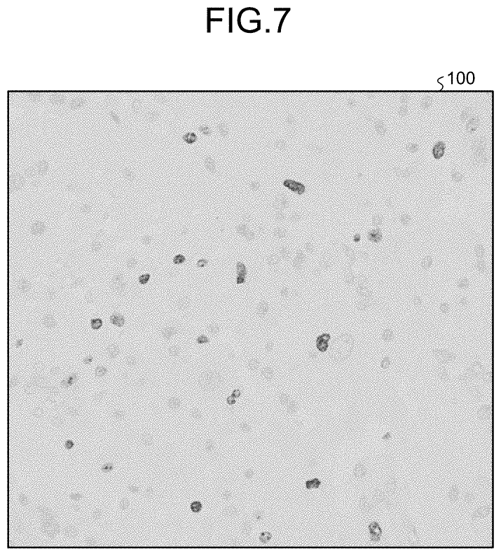

FIGS. 7 and 8 are diagrams illustrating the training image 200. Specifically, FIG. 7 illustrates an original image 100 (a pathological specimen image) obtained by capturing an image of the pathological specimen S on which immunostaining has been performed. FIG. 8 illustrates the training image 200 that is prepared based on the original image 100.

In the first embodiment, the training image 200 is an image that is labeled by a single pathologist with respect to each of various areas based on the original image 100. Specifically, the exemplary training image 200 illustrated in FIG. 8 is exemplary immunostaining and is an image in which positive cells PC and negative cells NC are labeled (marked) independently. Cells in interstitial areas (interstitial cells), lymphocytes, blood cells, etc., are not used for diagnosis and thus are not to be labelled. Dusts, etc., are also not to be labelled. There is a cell on which it is difficult to determine whether the cell is a positive cell PC, a negative cell NC, or a cell not to be labelled. It can be considered that this is because of a defect in the specimen, a defect in the process of preparing the sample, a defect in imaging, or because which type of cell the cell is cannot be determined from a two-dimensional image. This is also because the standards for classifying positive cells PC and negative cells NC are not digitally quantified and therefore cells cannot be digitally classified according to the depth of color in a boundary area between positive cells PC and negative cells NC. To deal with this, in the first embodiment, cells on which it is difficult to make the determination are labelled as diagnosis ambiguous areas ArA. Interstitial cells, lymphocytes, blood cells, etc., that are not used for diagnosis may be also labelled as diagnosis ambiguous areas ArA.

After step S1, the diagnosis ambiguous area learning unit 361 learns diagnosis ambiguous areas ArA by machine learning based on the acquired training image 200 (step S2: Learning a diagnosis ambiguous area). The training image 200 used for machine learning is not limited to a single image, and multiple images may be used.

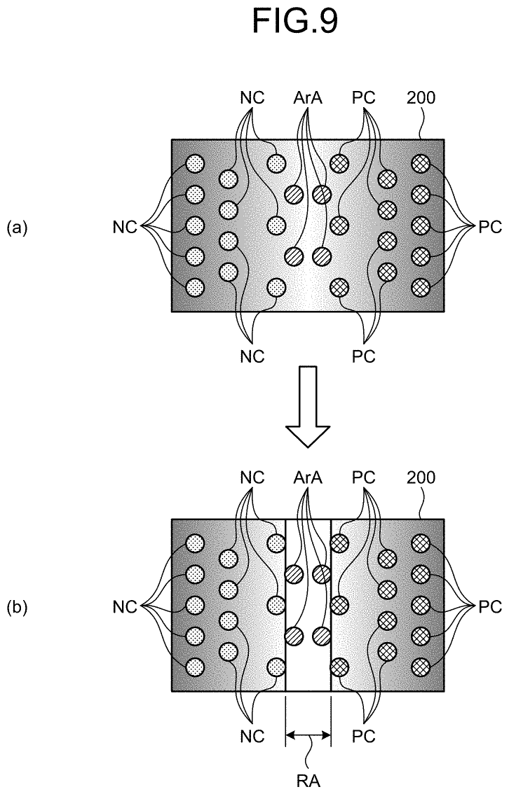

FIG. 9 is a diagram illustrating step S2. Specifically, in (a) and (b) of FIG. 9, the horizontal axis represents color feature data, such as the color space and the volume of pigment. (a) and (b) of FIG. 9 represent diagrams on which the positive cells PC, the negative cells NC, and the diagnosis ambiguous areas ArA in the training image 200 are transposed in corresponding positions on the horizontal axis (color feature data). In (a) and (b) of FIG. 9, the vertical axis has no meaning.

Specifically, at step S2, as illustrated in (a) of FIG. 9, the diagnosis ambiguous area learning unit 361 recognizes the positions of the diagnosis ambiguous areas ArA on the horizontal axis (color feature data) based on the training image 200. The diagnosis ambiguous area learning unit 361 learns the diagnosis ambiguous areas ArA by machine learning, thereby finding out a range RA ((b) of FIG. 9) on the horizontal axis (color feature data) covering the diagnosis ambiguous areas ArA. The diagnosis ambiguous area learning unit 361 stores the result of machine learning (the range RA, etc.,) in the learning result storage 334.

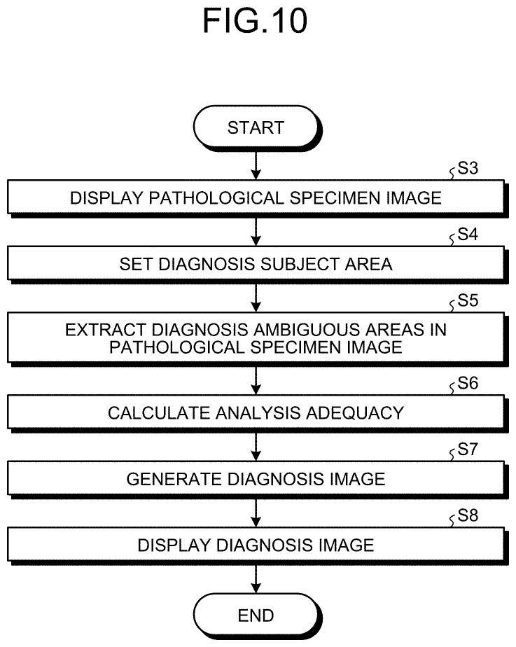

FIG. 10 is a flowchart illustrating the pathological specimen image processing method.

It is presupposed below that imaging the pathological specimen S performed by the imaging device 2 has completed and the pathological specimen image obtained by the imaging is already stored in the image data storage 332.

First of all, the controller 32 reads the pathological specimen image to be diagnosed that is stored in the image data storage 332 and causes the display unit 35 to display the pathological specimen image (step S3). The diagnosis area setting unit 362 sets a diagnosis subject area to be diagnosed in the pathological specimen image (the pathological specimen image displayed on the display unit 35) (step S4).

After step S4, the diagnosis ambiguous area extractor 363 extracts the diagnosis ambiguous areas in the pathological specimen image (the pathological specimen image displayed on the display unit 35) based on the result of machine learning that is stored in the learning result storage 334 (step S5: Extracting a diagnosis ambiguous area). For example, in the example in FIG. 9, the diagnosis ambiguous area extractor 363 extracts areas having color feature data (the color space and the volume of pigment) of the range RA in the pathological specimen image as diagnosis ambiguous areas.

After step S5, the analysis adequacy calculator 364 calculates an analysis adequacy of the diagnosis subject area based on the diagnosis ambiguous areas that are extracted at step S5 (step S6).

After step S6, the image generator 365 generates a diagnosis image that enables the diagnosis ambiguous areas, which are extracted at step S5, in the pathological specimen image to be distinguished from other areas and that contains an analysis adequacy image corresponding to the analysis adequacy, which is calculated at step S6 (step S7: Generating an image). The controller 32 causes the display unit 35 to display the diagnosis image (step S8).

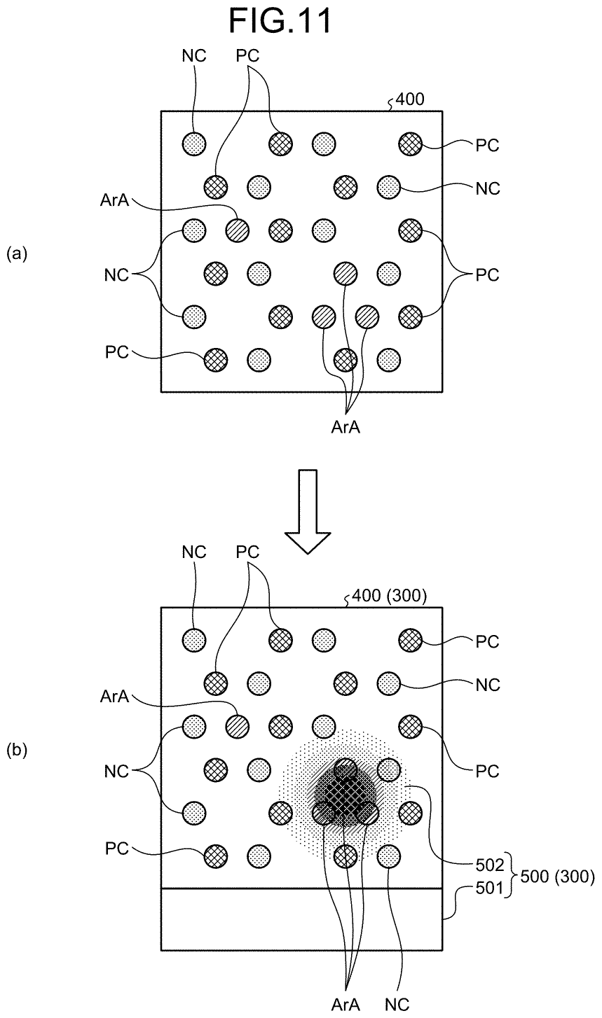

FIGS. 11 to 15 are diagrams of an exemplary diagnosis image 300.

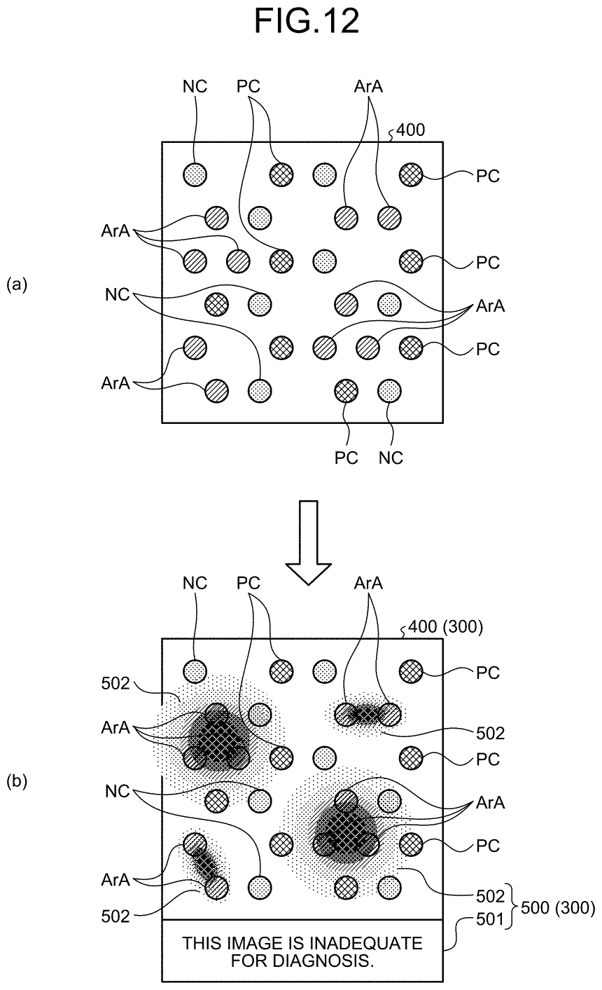

First of all, the diagnosis image 300 that is exemplified in FIGS. 11 and 12 will be described.

FIGS. 11 and 12 illustrate the case where the diagnosis area setting unit 362 sets a plurality of areas obtained by dividing the pathological specimen image for diagnosis subject areas, respectively, at step S4. FIG. 11 illustrates the case where few diagnosis ambiguous areas ArA are extracted at step S5. FIG. 12 illustrates the case where many diagnosis ambiguous areas ArA are extracted at step S5.

At step S7, first of all, as illustrated in (a) of FIG. 11 and (a) of FIG. 12, the image generator 365 generates a distinguishing image 400 that enables the diagnosis ambiguous areas ArA that are extracted at step S5 to be distinguished from other areas in the pathological specimen image. In the examples in (a) of FIG. 11 and (a) of FIG. 12, the diagnosis subject images ArA are hatched to be distinguishable from other areas. As illustrated in (b) of FIG. 11 and (b) of FIG. 12, the image generator 365 generates the diagnosis image 300 obtained by synthesizing an analysis adequacy image 500 with the distinguishing image 400 based on the analysis adequacy that is calculated at step S6.

As illustrated in (b) of FIG. 11 or (b) of FIG. 12, the analysis adequacy image 500 includes a message image 501 and a superimposed image 502.

The message image 501 is an image containing a message, such as “This image is inadequate for diagnosis.”, when the average of analysis adequacies that are calculated respectively for the diagnosis subject areas at step S6 is above a reference value. In the example illustrated in (b) of FIG. 11, the message image 501 is blank because the average of analysis adequacies of the diagnosis subject areas is under the reference value. On the other hand, in the example illustrated in (b) of FIG. 12, the aforementioned message is written in the message image 501 because the average of analysis adequacies of the diagnosis subject areas is above the reference value.

The superimposed image 502 is an image that enables the diagnosis subject areas whose corresponding analysis adequacies, which are calculated at step S6, are above the reference value among the diagnosis subject areas from other areas. In the examples of (b) of FIG. 11 and (b) of FIG. 12, the superimposed image 502 is a heat map image and is superimposed on the diagnosis subject area whose corresponding analysis adequacy is above the reference value.

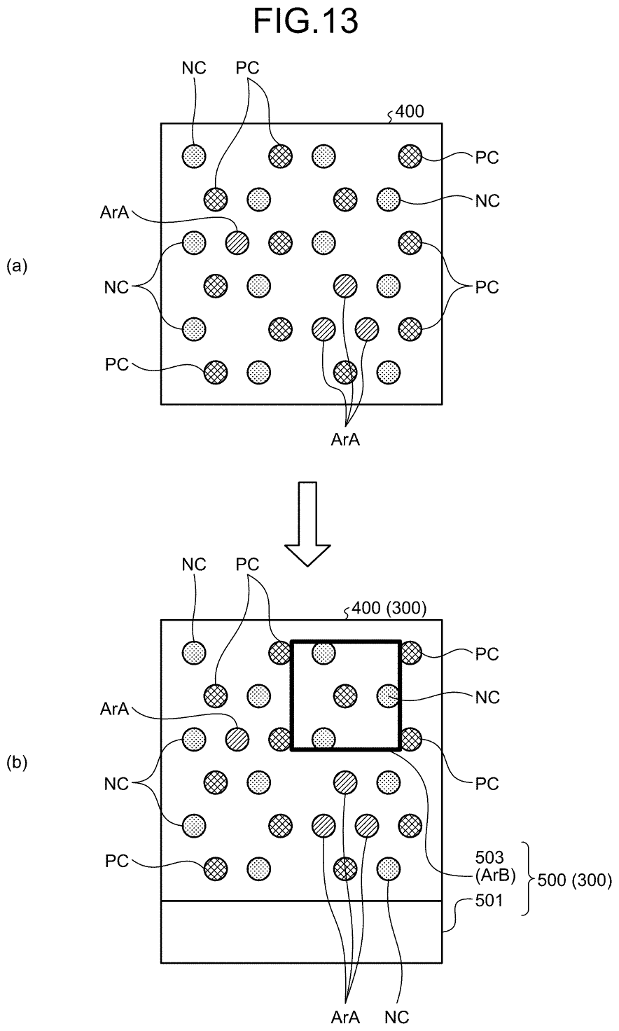

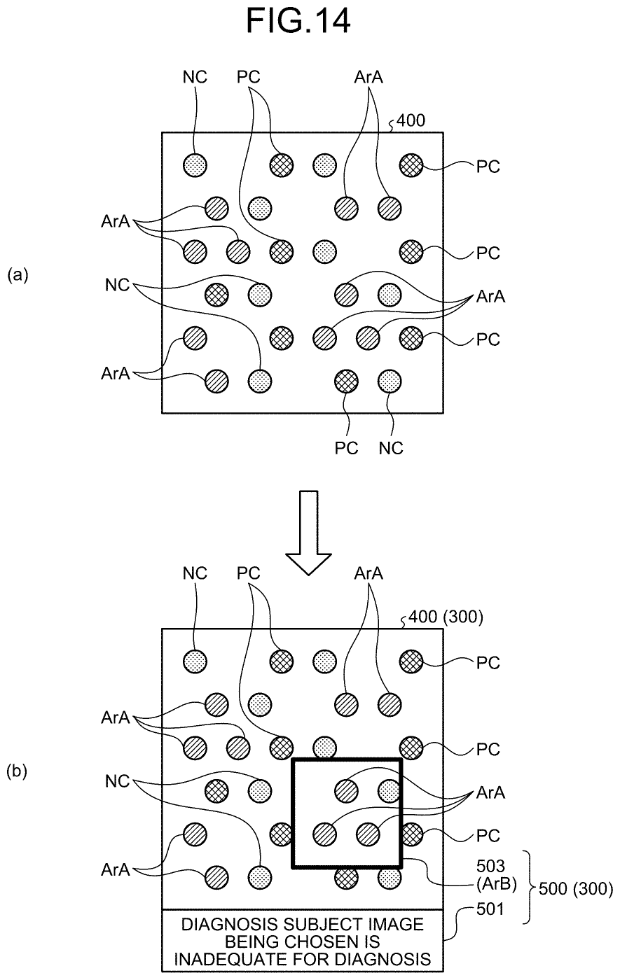

The diagnosis image 300 exemplified in FIGS. 13 and 14 will be described.

FIGS. 13 and 14 illustrate the case where the input unit 34 receives an operation of specifying a diagnosis subject area in the pathological specimen image that is displayed on the display unit 35, which is the operation performed by the user, and the diagnosis area setting unit 362 sets part of the pathological specimen image corresponding to the specifying operation for a diagnosis subject area ArB at step S4. In other words, the input unit 34 corresponds to an operation receiver according to the disclosure. In the examples in (b) of FIG. 13 and (b) of FIG. 14, the diagnosis subject area ArB is represented by a rectangular frame.

At step S7, first of all, as illustrated in (a) of FIG. 13 and (a) of FIG. 14, the image generator 365 generates the same distinguishing image 400 as that in (a) of FIG. 11 or (a) of FIG. 12. As illustrated in (b) of FIG. 13 and (b) of FIG. 14, the image generator 365 generates the diagnosis image 300 obtained by synthesizing the analysis adequacy image 500 with the distinguishing image 400 based on the analysis adequacy that is calculated at step S6.

As illustrated in (b) of FIG. 13 and (b) of FIG. 14, the analysis adequacy image 500 includes a rectangular frame image 503 and the message image 501.

The rectangular frame image 503 is an image of a rectangular frame representing the diagnosis subject area ArB and is superimposed on the distinguishing image 400 in the position corresponding to the diagnosis subject area ArB that is set at step S4 according to the specifying operation made by the user on the input unit 34.

The message image 501 is an image containing a message, such as “The diagnosis subject area being chosen is inadequate for diagnosis.”, when the analysis adequacy that is calculated for the diagnosis subject areas ArB, which is specified by the user, at step S6 is above the reference value. In the example illustrated in (b) of FIG. 13, the message image 501 is blank because the analysis adequacy of the diagnosis subject area ArB is under the reference value. On the other hand, in the example illustrated in (b) of FIG. 14, the aforementioned message is written in the message image 501 because the analysis adequacy of the diagnosis subject area ArB is above the reference value.

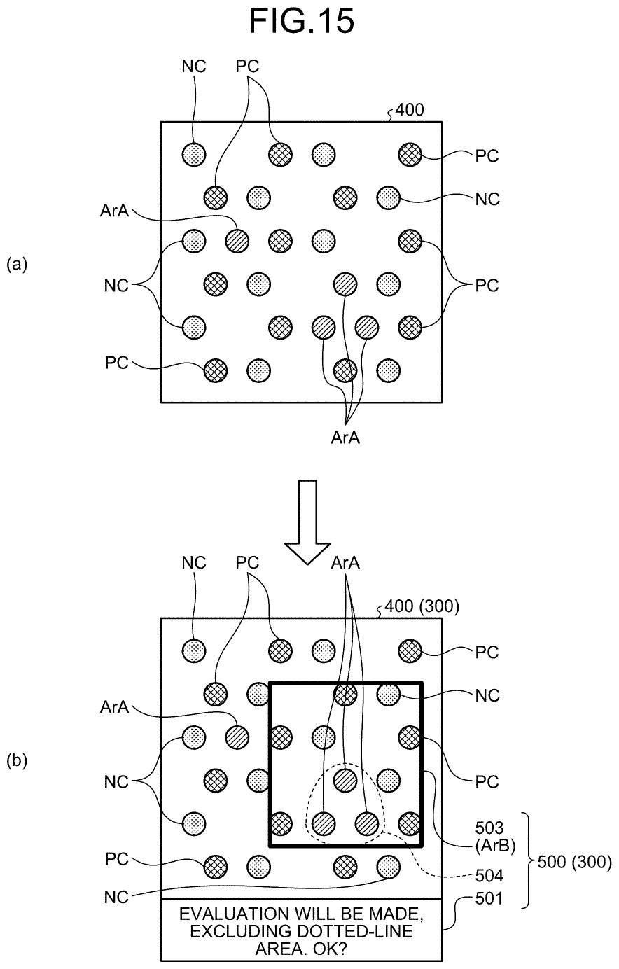

Lastly, the diagnosis image 300 exemplified in FIG. 15 will be described.

As FIGS. 13 and 14 do, FIG. 15 illustrates the case where the input unit 34 receives an operation of specifying a diagnosis subject area in the pathological specimen image that is displayed on the display unit 35, which is the operation performed by the user, and the diagnosis area setting unit 362 sets part of the pathological specimen image corresponding to the specifying operation for the diagnosis subject area ArB at step S4.

At step S7, first of all, as illustrated in (a) of FIG. 15, the image generator 365 generates the same distinguishing image 400 as that in (a) of FIG. 11 or (a) of FIG. 12. As illustrated in (b) of FIG. 15, the image generator 365 generates the diagnosis image 300 obtained by synthesizing the analysis adequacy image 500 with the distinguishing image 400 based on the analysis adequacy that is calculated at step S6.

As illustrated in (b) of FIG. 15, the analysis adequacy image 500 includes the rectangular frame image 503, an excluded area image 504, and the message image 501.

The rectangular frame image 503 is the same image as that in (b) of FIG. 13 or (b) of FIG. 14.

The excluded area image 504 is an image that is superimposed over the whole diagnosis ambiguous areas ArA in the diagnosis subject are ArB when the analysis adequacy that is calculated at step S6 for the diagnosis subject area ArB specified by the user is above a reference value. In the example in FIG. 15, the exterior edge of the excluded area image 504 is represented by the dotted line.

The message image 501 is an image containing, for example, a message “Evaluation will be made excluding the dotted-line area. OK?”, or the like, when the analysis adequacy calculated at step S6 for the diagnosis subject area ArB specified by the user is above the reference value.

According to the first embodiment described above, the following effects are achieved.

The image processing apparatus 3 according to the first embodiment performs learns the diagnosis ambiguous areas ArA by machine learning based on the training image 200. The image processing apparatus 3 then extracts the diagnosis ambiguous areas ArA in the pathological specimen image based on the result of machine learning and generates and displays the diagnosis image 300 that enables the extracted diagnosis ambiguous areas ArA to be distinguished from other areas.

This enables the user to recognize areas inadequate for diagnosis. In other words, the user performs classification by sight and this allows diagnosis on appropriate areas.

Accordingly, the image processing apparatus 3 according to the first embodiment achieves an effect that it is possible to give an appropriate diagnosis.

The image processing apparatus 3 according to the first embodiment learns the diagnosis ambiguous areas ArA by machine learning based on the training image 200 where the diagnosis ambiguous areas ArA are marked in advance.

This enables appropriate machine learning for areas on which diagnoses made by a single pathologist are ambiguous (diagnosis ambiguous areas ArA).

The image processing apparatus 3 according to the first embodiment calculates analysis adequacies based on the extracted diagnosis ambiguous areas ArA. The image processing apparatus 3 generates and displays the diagnosis image 300 containing the distinguishing image 400 that enables the extracted diagnosis ambiguous areas ArA to be distinguished from other areas and the analysis adequacy image 500 corresponding to the analysis adequacies.

This enable the user to clearly recognize areas inadequate for diagnosis (analysis).

Specifically, FIGS. 11 and 12 exemplify the diagnosis image 300 that is displayed before the operation of specifying the diagnosis subject area ArB, which is performed by the user on the input unit 34. Displaying the diagnosis image 300 allows the user to check the analysis adequacy of each area in the pathological specimen image before specifying the diagnosis subject area ArB. In the examples in FIGS. 13 to FIG. 15, the user is able to check in real time the analysis adequacy of the diagnosis subject area ArB that is specified by the user.

A second embodiment will be described.

In the following description, the same components and steps as those of the above-described first embodiment are denoted with like reference numbers and detailed description thereof will be omitted or simplified.

The second embodiment differs from the above-described first embodiment only in the method of learning diagnosis ambiguous areas ArA by machine learning that is performed by the diagnosis ambiguous area learning unit 361.

The method of learning diagnosis ambiguous areas ArA by machine learning according to the second embodiment will be described below.

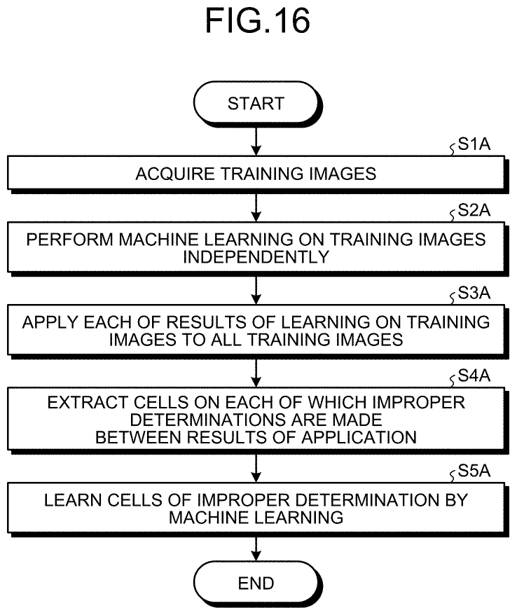

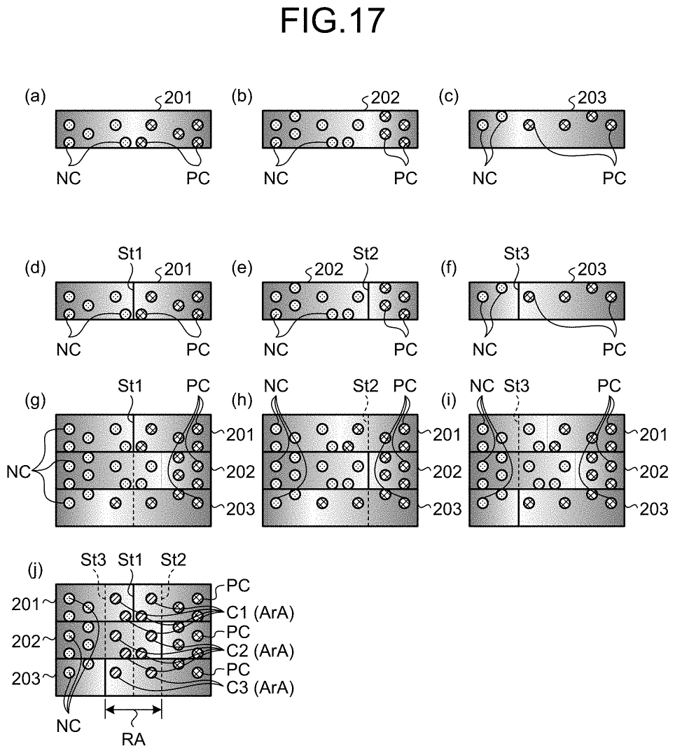

FIG. 16 is a flowchart illustrating the method of learning diagnosis ambiguous areas ArA by machine learning according to the second embodiment. FIG. 17 is a diagram illustrating the method of learning diagnosis ambiguous areas ArA by machine learning illustrated in FIG. 16. Specifically, FIG. 17 is a diagram corresponding to FIG. 9.

First of all, the diagnosis ambiguous area learning unit 361 reads a plurality of training images 201 to 203 ((a) to (c) of FIG. 17) that are stored in the training image storage 333 (step S1A).

In the above-described first embodiment, the training image 200 is prepared (each of various areas is labeled based on an original image) by the single pathologist. On the other hand, in the second embodiment, the training images 201 to 203 ((a) to (c) of FIG. 17) are prepared (each of various areas is labelled in advance based on the original images) by a plurality of (three in the example in FIG. 17) medical facilities or a plurality of (three in the example in FIG. 17), respectively. As illustrated in (a) to (c) of FIG. 17, in the training images 201 to 203 according to the second embodiment, different from the training image 200 illustrated in the above-described first embodiment, only positive cells PC and negative cells NC are labelled (diagnosis ambiguous areas ArA are not labelled). The medical facilities or pathologists having made the training images 201 to 203 illustrated in (a) to (c) of FIG. 17 are different from one another. In other words, the training images 201 to 203 according to the second embodiment are prepared according to a plurality of different standards St1 to St3 ((d) to (f) of FIG. 17).

After step S1A, the diagnosis ambiguous area learning unit 361 performs machine learning independently on the acquired training images 201 to 203 (step S2A).

Specifically, at step S2A, as illustrated in (d) to (f) of FIG. 17, the diagnosis ambiguous area learning unit 361 recognizes positions of positive cells PC and negative cells NC on the horizontal axis (color feature data) based on the training images 201 to 203. The diagnosis ambiguous area learning unit 361 then performs machine learning on the training images 201 to 203 independently, thereby finding out the standards St1 to St3 of the respective medical facilities or pathologist having determined the positive cells PC and the negative cells NC. The diagnosis ambiguous area learning unit 361 stores the results of machine learning (the standards St1 to St3, etc.) in the learning result storage 334.

Each of the training images 201 to 203 used for machine learning is not limited to a single image, and a plurality of mages may be used.

After step S2A, the diagnosis ambiguous area learning unit 361 applies each of the learning results (standards St1 to St3) acquired at step S2A to all the training images 201 to 203 (step S3A).

Specifically, at step S3A, as illustrated in (g) of FIG. 17, the diagnosis ambiguous area learning unit 361 determines positive cells PC and negative cells NC according to the standard St1 with respect to the training images 201 to 203. As illustrated in (h) of FIG. 17, the diagnosis ambiguous area learning unit 361 also determines positive cells PC and negative cells NC according to the standard St2 with respect to the training images 201 to 203. As illustrated in (i) of FIG. 17, the diagnosis ambiguous area learning unit 361 further determines positive cells PC and negative cells NC according to the standard St3 with respect to the training images 201 to 203.

After step S3A, the diagnosis ambiguous area learning unit 361 extracts areas on each of which different determinations are made between at least two of a plurality of results of application at step S3A (cells of improper determination) (step S4A).

Specifically, in the training image 201, as illustrated in (j) of FIG. 17, cells C1 are determined as negative cells NC according to the standard St2 but are determined as positive cells PC according to the standard St3. In the training image 202, cells C2 are determined as negative cells PC according to the standard St2 but are determined as positive cells PC according to the standard St3. Furthermore, in the training image 203, cells C3 are determined as negative cells NC according to the standard St2 but are determined as positive cells PC according to the standard St3. Accordingly, at step S4A, the diagnosis ambiguous area learning unit 361 extracts the cells C1 to C3 as cells of improper determination. The cells C1 to C3 of improper determination correspond to diagnosis ambiguous areas ArA.

After step S4A, the diagnosis ambiguous area learning unit 361 learns by machine learning the cells of improper determination that are extracted at step S4A (step S5A: Learning a diagnosis ambiguous area).

Specifically, at step S5A, based on the training images 201 to 203, the diagnosis ambiguous area learning unit 361 recognizes the positions of the cells C1 to C3 of improper determination, which are extracted at step S4A, on the horizontal axis (color feature data). The diagnosis ambiguous area learning unit 361 then performs machine learning on the cells C1 to C3 of improper determination, thereby finding out the range RA ((j) of FIG. 17) on the horizontal axis (color feature data) covering the cells C1 to C3 of improper determination. The diagnosis ambiguous area learning unit 361 then stores the result of machine learning (the range RA, etc.) in the learning result storage 334.

According to the second embodiment described above, the following effects are achieved in addition to the same effects as those of the above-described first embodiment.

The image processing apparatus 3 according to the second embodiment performs machine learning independently on the training images 201 to 203 that are prepared respectively according to the different standards St1 to St3 and applies the results of machine learning to all the training images 201 to 203. The image processing apparatus 3 learns the diagnosis ambiguous areas ArA by machine learning based on the areas on each of which different determinations are made between at least two of the results of application.

For this reason, it is possible to appropriately learn by machine learning the areas (the diagnosis ambiguous areas AraA) on each of which different diagnoses are given by the medical facilities or pathologists, respectively.

A third embodiment will be described.

In the following description, the same components and steps as those of the above-described first embodiment are denoted with like reference numbers and detailed description thereof will be omitted or simplified.

The third embodiment differs from the above-described first embodiment only in the method of learning diagnosis ambiguous areas ArA by machine learning that is performed by the diagnosis ambiguous area learning unit 361.

The method of learning diagnosis ambiguous areas ArA by machine learning according to the third embodiment will be described below.

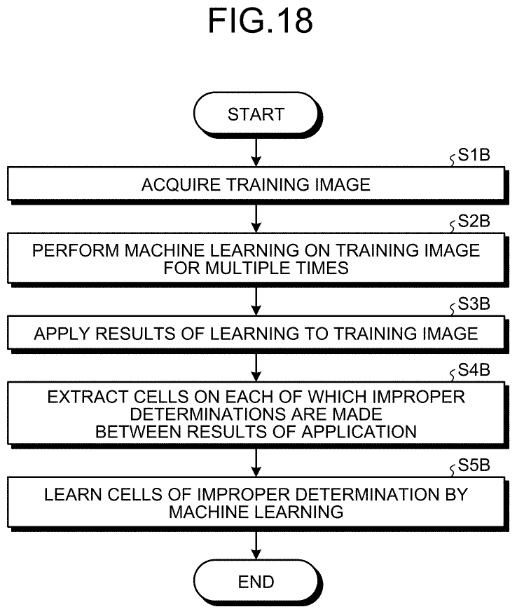

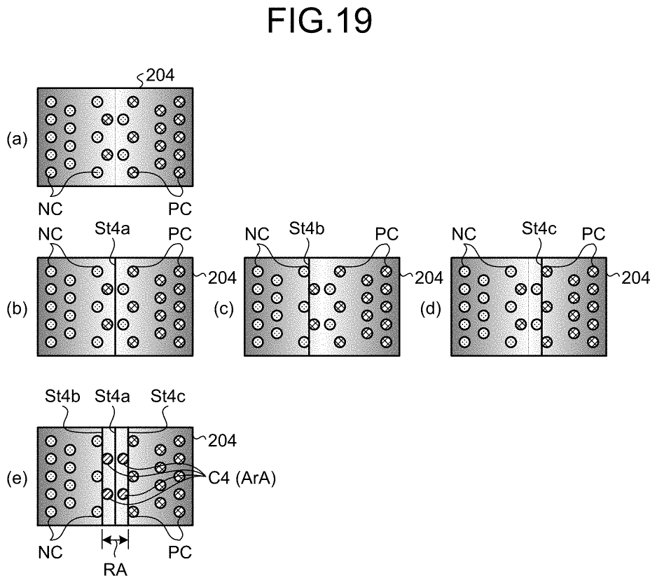

FIG. 18 is a flowchart illustrating the method of learning diagnosis ambiguous areas ArA by machine learning according to the third embodiment. FIG. 19 is a diagram illustrating the method of learning diagnosis ambiguous areas ArA by machine learning illustrated in FIG. 18. Specifically, FIG. 19 is a diagram corresponding to FIG. 9.

First of all, the diagnosis ambiguous area learning unit 361 reads a training image 204 ((a) of FIG. 19) that is stored in the training image storage 333 (step S1B).

As in the above-described first embodiment, the training image 204 according to the third embodiment is an image that is prepared (each of various areas is labeled based on an original image) by a single pathologist. In other words, the training image 204 according to the third embodiment is prepared according to a single standard. In the training image 204 according to the third embodiment, as illustrated in (a) of FIG. 19, different from the training image 200 described in the first embodiment, only positive cells PC and negative cells NC are labelled (diagnosis ambiguous areas ArA are not labelled).

After step S1B, the diagnosis ambiguous area learning unit 361 performs machine learning on the acquired training image 204 for multiple times (step S2B).

Specifically, at step S2B, based on the training image 204, the diagnosis ambiguous area learning unit 361 recognizes the positions of the positive cells PC and negative cells NC on the horizontal axis (color feature data). The diagnosis ambiguous area learning unit 361 then performs machine learning for the first time, thereby finding out a standard St4a ((b) of FIG. 19) of the single pathologist having determined the positive cells PC and negative cells NC. The diagnosis ambiguous area learning unit 361 then performs machine learning for the second time, thereby finding out a standard St4b ((c) of FIG. 19) of the single pathologist having determined the positive cells PC and negative cells NC. The diagnosis ambiguous area learning unit 361 then performs machine learning for the third time, thereby finding out a standard St4c ((d) of FIG. 19) of the single pathologist having determined the positive cells PC and negative cells NC. The diagnosis ambiguous area learning unit 361 stores the results of machine learning (the standards St4a to St4c) in the learning result storage 334.

The training image used for machine learning is not limited to a single image, and multiple images may be used.

After step S2B, the diagnosis ambiguous area learning unit 361 applies the results of learning (the standards St4a to St4c) to the training image 204 (step S3B).

Specifically, at step S3B, as illustrated in (b) of FIG. 19, the diagnosis ambiguous area learning unit 361 determines positive cells PC and negative cells NC according to the standard St4a with respect to the training image 204. As illustrated in (c) of FIG. 19, the diagnosis ambiguous area learning unit 361 determines positive cells and negative cells NC according to the standard St4b with respect to the training image 204. As illustrated in (c) of FIG. 19, the diagnosis ambiguous area learning unit 361 further determines positive cells PC and negative cells NC according to the standard St4b with respect to the training image 204. As illustrated in (d) of FIG. 19, the diagnosis ambiguous area learning unit 361 further determines positive cells and negative cells NC according to the standard St4c with respect to the training image 204.

After step S3B, the diagnosis ambiguous area learning unit 361 extracts areas (cell of improper determination) on each of which different determinations are made between at least two of a plurality of results of application at step S3B (step S4B).

Specifically, in the training image 204, as illustrated in (e) of FIG. 19, cells C4 are determined as positive cells PC according to the standard St4b but are determined as negative cells NC according to the standard St4c. Accordingly, at step S4B, the diagnosis ambiguous area learning unit 361 extracts the cells C4 as cells of improper determination. The cells C4 of improper determination correspond to the diagnosis ambiguous areas ArA.

After step S4B, the diagnosis ambiguous area learning unit 361 performs machine learning on the cells of improper determination that are extracted at step S4B (step S5B: Learning a diagnosis ambiguous area).

Specifically, at step S5B, based on the training image 204, the diagnosis ambiguous area learning unit 361 recognizes the positions of the cells C4 of improper determination that are extracted at step S4B on the horizontal axis (color feature data). The diagnosis ambiguous area learning unit 361 then performs machine learning on the cells C4 of improper determination, thereby finding out a range RA ((e) of FIG. 19) on the horizontal axis (color feature data) covering the cells C4 of improper determination. The diagnosis ambiguous area learning unit 361 then stores the result of machine learning (the range RA, etc.) in the learning result storage 334.

According to the third embodiment described above, the following effects are achieved in addition to the same effects as those of the above-described first embodiment.

The image processing apparatus 3 according to the third embodiment performs machine learning for multiple times on the training image 204 that is prepared based on the single standard and applies each of the results of machine learning to the training image 204. The image processing apparatus 3 performs learns by machine learning the diagnosis ambiguous areas ArA based on the areas on each of which different determinations are made between at least two of the results of application.

For this reason, when the machine learning has random features, it is possible to appropriately learn by machine learning the diagnosis ambiguous areas ArA caused by the features.

A fourth embodiment will be described.

In the following description, the same components and steps as those of the above-described first embodiment are denoted with like reference numbers and detailed description thereof will be omitted or simplified.

The fourth embodiment differs from the above-described first embodiment only in the method of learning diagnosis ambiguous areas ArA by machine learning that is performed by the diagnosis ambiguous area learning unit 361.

The method of learning diagnosis ambiguous areas ArA by machine learning according to the fourth embodiment will be described below.

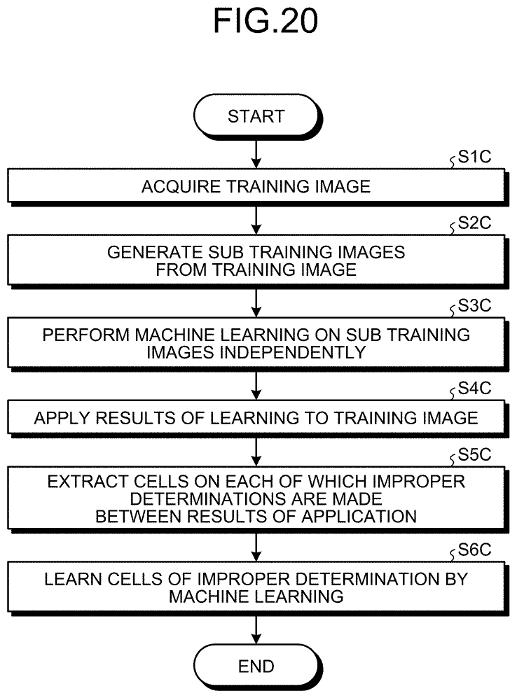

FIG. 20 is a flowchart illustrating the method of learning diagnosis ambiguous areas ArA by machine learning according to the fourth embodiment. FIG. 20 is a diagram illustrating the method of learning diagnosis ambiguous areas ArA by machine learning illustrated in FIG. 19. Specifically, FIG. 20 is a diagram corresponding to FIG. 9.

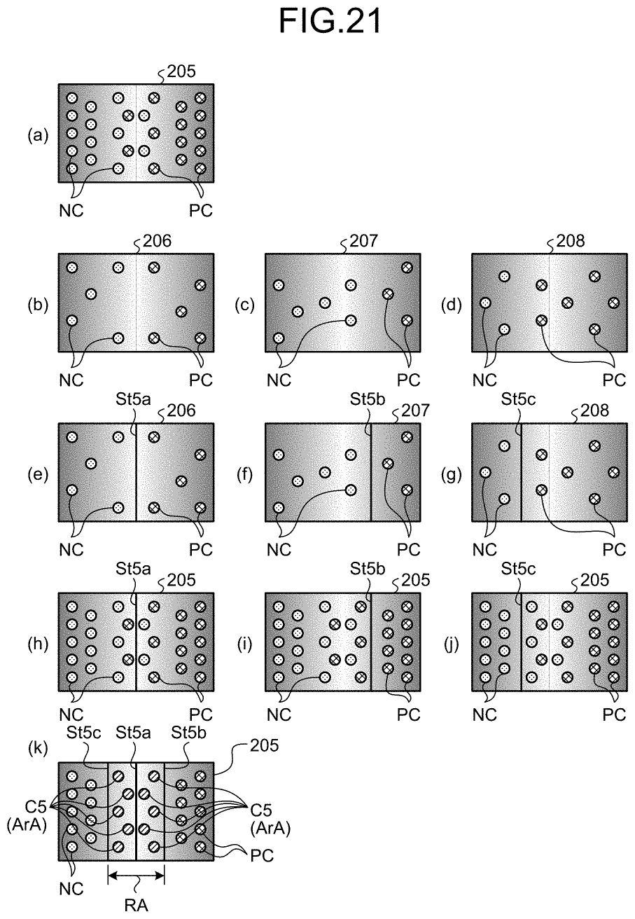

First of all, the diagnosis ambiguous area learning unit 361 reads a training image 205 ((a) of FIG. 21) that is stored in the training image storage 333 (step S1C).

As in the above-described first embodiment, the training image 205 according to the fourth embodiment is an image that is prepared (each of various areas is labeled based on an original image) by a single pathologist. In other words, the training image 205 according to the fourth embodiment is prepared according to a single standard. In the training image 205 according to the fourth embodiment, as illustrated in (a) of FIG. 21, different from the training image 200 described in the above-described first embodiment, only positive cells PC and negative cells NC are labelled (diagnosis ambiguous areas ArA are not labelled).

After step S1C, the diagnosis ambiguous area learning unit 361 generates a plurality of (three in the fourth embodiment) different sub training images 206 to 208 ((b) to (d) of FIG. 21) obtained by thinning data (positive cells PC and negative cells PC) out of the acquired training image 205 (step S2C).

After step S2C, the diagnosis ambiguous area learning unit 361 performs machine learning independently on the sub training images 206 to 208 that are generated at step S2C (step S3C).

Specifically, at step S3C, the diagnosis ambiguous area learning unit 361 recognizes the positions of the positive cells PC and negative cells NC on the horizontal axis (color feature data) based on the sub training image 206. The diagnosis ambiguous area learning unit 361 performs machine learning on the sub training image 206, thereby finding out a standard St5a ((e) of FIG. 21) to determine positive cells PC and negative cells NC. The diagnosis ambiguous area learning unit 361 similarly performs machine learning on a sub-training image 207, thereby finding out a standard St5b ((f) of FIG. 21) to determine positive cells PC and negative cells NC. The diagnosis ambiguous area learning unit 361 similarly performs machine learning on the sub training images 208, thereby finding out a standard St5c ((g) of FIG. 21) to determine positive cells PC and negative cells NC. The diagnosis ambiguous area learning unit 361 then stores results of machine learning (standards St5a to St5c) in the learning result storage 334.

The training image 205 used for machine learning is not limited to a single image, and a plurality of images may be used.

After step S3C, the diagnosis ambiguous area learning unit 361 applies the results of learning (the standards St5a to St5c) to the training image 205 (step S4C).

Specifically, at step S4C, as illustrated in (h) of FIG. 21, the diagnosis ambiguous area learning unit 361 determines positive cells PC and negative cells NC according to the standard St5a with respect to the training image 205. As illustrated in (i) of FIG. 21, the diagnosis ambiguous area learning unit 361 determines positive cells PC and negative cells NC according to the standard St5b with respect to the training image 205. As illustrated in (j) of FIG. 21, the diagnosis ambiguous area learning unit 361 determines positive cells PC and negative cells NC according to the standard St5c with respect to the training image 205.

After step S4C, the diagnosis ambiguous area learning unit 361 extracts areas (cells of improper determination) on each of which different determinations are made between at least two of a plurality of results of application at step S4C (step S5C).

Specifically, in the training image 205, as illustrated in (k) of FIG. 21, cells C5 are determined as negative cells NC according to the standard St5b but are determined as positive cells PC according to the standard St5c. Accordingly, at step SSC, the diagnosis ambiguous area learning unit 361 extracts the cells C5 as cells of improper determination. The cells 5 of improper determination correspond to the diagnosis ambiguous areas ArA.

After step SSC, the diagnosis ambiguous area learning unit 361 performs machine learning on the cells of improper determination that are extracted at step S5C (step S6C: Learning a diagnosis ambiguous area).

Specifically, at step S6C, based on the training image 205, the diagnosis ambiguous area learning unit 361 recognizes the positions of the cells C5 of improper determination that are extracted at step S5C on the horizontal axis (color feature data). The diagnosis ambiguous area learning unit 361 then performs machine learning on the cells C5 of improper determination, thereby finding out a range RA ((k) of FIG. 21) on the horizontal axis (color feature data) covering the cells C5 of improper determination. The diagnosis ambiguous area learning unit 361 then stores the result of machine learning (the range RA, etc.) in the learning result storage 334.

According to the fourth embodiment described above, the following effects are achieved in addition to the same effects as those of the first embodiment.

The image processing apparatus 3 according to the fourth embodiment performs machine learning independently on the different sub training images 206 to 208 obtained by randomly thinning data out of the training image 205 that is prepared based on a single standard and applies each of the results of machine learning to the training images 205. The image processing apparatus 3 learns the diagnosis ambiguous areas ArA by machine learning based on the areas on each of which different determinations are made between at least two of the results of application.

For this reason, it is possible to appropriately learn by machine learning areas (diagnosis ambiguous areas AraA) on which diagnosis tends to vary depending on the magnitude of data volume of the training image (the magnitude in the number of positive cells PC and negative cells NC).

Modes for carrying out the disclosure have been described; however, the disclosure should not be limited to only the above-described first to fourth embodiments.

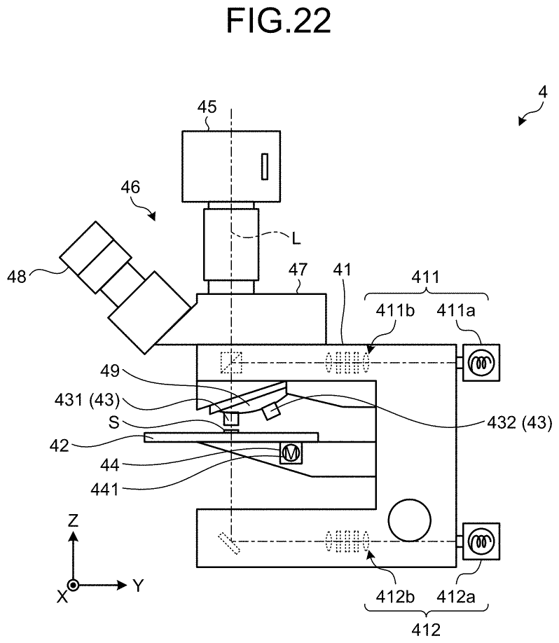

FIG. 22 is a diagram illustrating a modification of the first to fourth embodiments.

In the first to fourth embodiments, a microscope device 4 illustrated in FIG. 22 may be used as the imaging device according to the disclosure.

The microscope device 4 includes an approximately C-shaped arm 41 including an epi-illumination unit 411 and a transillumination unit 412 that are provided therein; a specimen stage 42 on which the pathological specimen S is placed; an objective lens 43 that is provided on one side of a lens tube 46 via a trinocular tube unit 47 such that the objective lens 43 is opposed to the specimen stage 42; a stage position changer 44 that moves the specimen stage 42, and an imaging unit 45.

A configuration including the image forming optical system 23, the filter unit 25, and the RGB camera 24 that are described in the above-described first to fourth embodiments can be exemplified as the imaging unit 45.

The trinocular tube unit 47 diverges observation light from a pathological specimen S that is incident on the objective lens 43 to the imaging unit 45 that is provided on the other end of the lens tube 46 and to an eyepiece lens unit 48 for the user to directly observe the pathological specimen S.

The epi-illumination unit 411 corresponds to an illuminator according to the disclosure. The epi-illumination unit 411 includes an epi-illumination light source 411a and an epi-illumination optical system 411b and applies epi-illumination light to the pathological specimen S. The epi-illumination optical system 411b includes various optical members (a filter unit, a shutter, a field stop, an aperture stop, etc.) that focuses the illumination light that is emitted from the epi-illumination light source 411a and guides the focused illumination light in a direction of an observation optical path L.

The transillumination unit 412 corresponds to the illuminator according to the disclosure. The transillumination unit 412 includes a transillumination light source 412a and a transillumination optical system 412b and applies transillumination light to the pathological specimen S. The transillumination optical system 412b includes various optical members (a shutter, a field stop, an aperture stop, etc.) that focuses the illumination light that is emitted from the transillumination light source 412a and guides the focused illumination light in the direction of the observation optical path L.

The objective lens 43 is attached to a revolver 49 that is able to hold a plurality of objective lenses (for example, objective lenses 431 and 432) whose magnitudes are different from each other. Switching between the objective lenses 431 and 432 that are opposed to the specimen stage 42 by rotating the revolver 49 makes it possible to change the imaging magnification.

In the lens tube 46, a plurality of zoom lenses and a zoom unit including a driver that changes the positions of the zoom lenses are provided. The zoom unit enlarges or reduces a subject image in the imaging field by adjusting the position of each of the zoom lenses.

The stage position changer 44 includes, for example, a driver 441, such as a stepping motor, and changes the imaging field by moving the specimen stage 42 within a X-Y plane and thus changing the position of the specimen stage 42. The stage position changer 44 adjusts the focal point of the objective lens 43 to the pathological specimen S by moving the specimen stage 42 along a Z-axis.

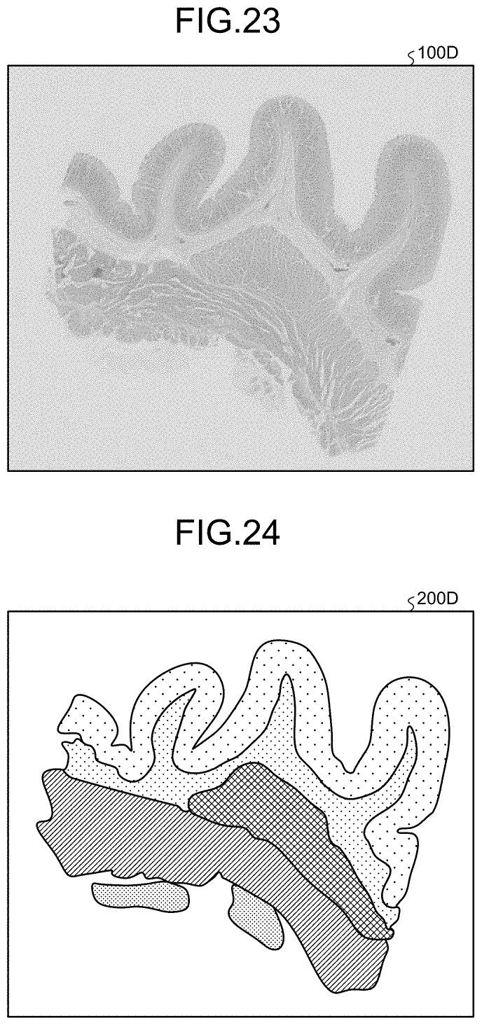

FIGS. 23 and FIG. 24 are diagrams illustrating a modification of the first to fourth embodiments. Specifically, FIG. 23 represents an original image 100D (pathological specimen image) on which HE staining has been performed. FIG. 24 represents a training image 200D that is prepared based on the original image 100D.

In the above-described first to fourth embodiments, the training images 200 to 205 that are prepared based on the original image 100 obtained by imaging the pathological specimen S on which immunostaining has been performed are exemplified as a training image according to the disclosure; however, the training image is not limited thereto. For example, the training image 200D (FIG. 24) that is prepared based on the original image 100D (FIG. 23) on which HE staining has been performed may be used as the training image according to the disclosure. The training image 200D is an image on which different labelling (a different mark) is applied to each area in the original image 100D.

The first to fourth embodiments may employ a configuration to, for the training images 200 to 205 in each of which each area is already labelled, change the labelling of at least one of the areas to other labelling (for example, the area that is labelled as a diagnosis ambiguous area ArA is re-labelled as a negative cell NC) and, based on the changed training image, perform machine learning again (additional learning). Furthermore, on the pathological specimen image used at step S3 to S8, it is determined whether the diagnosis ambiguous areas ArA that are extracted at step S5 are appropriate. The embodiments may employ a configuration to add, as a training image, the pathological image whose diagnosis ambiguous area ArA having been determined as an inappropriate area is labelled as another area and, based on the added training image, perform machine learning (additional learning) again. Furthermore, the result of learning may be managed by a cloud. The result of learning in the cloud may reflect the additional learning.

In the first to fourth embodiments, the color feature data is employed as the horizontal axis in FIGS. 9, 17, 19 and 21; however, the horizontal axis is not limited thereto. For example, mode feature data, such as particle feature data or texture feature data, may be employed. The horizontal axis may be color feature data and the vertical axis may be mode feature data. In other words, when machine learning is performed on the training images 200 to 205, at least one of color feature data and mode feature data is used. A configuration to perform machine learning using, in addition to color feature data and mode feature data, other feature data may be employed.

In the first to fourth embodiments, the diagnosis image 300 illustrated in FIGS. 11 to 15 is exemplified only. It suffices if an image that at least enables diagnosis ambiguous areas ArA to be distinguished from other areas be used. The analysis adequacy image 500 illustrated in FIGS. 13 to 15 is an example only. The message image 501 may be omitted and a configuration to, when the analysis adequacy of the diagnosis subject area ArB specified by the user, which is the analysis adequacy calculated at step S6, is above the reference value, change the mode of displaying the rectangular frame image 503 (change the color, hatch the rectangular frame image 503, or make the image blink) may be employed.

The image processing apparatus, the imaging system, the image processing method, and the image processing program achieve an effect that it is possible to give an appropriate diagnosis.

The above and other features, advantages and technical and industrial significance of this disclosure will be better understood by reading the following detailed description of presently preferred embodiments of the disclosure, when considered in connection with the accompanying drawings.

What is claimed is:

1. An image processing apparatus for processing a pathological specimen image obtained by imaging a pathological specimen, the image processing apparatus comprising a processor comprising hardware, the processor being configured to:
perform machine learning independently on a plurality of training images for machine learning that are prepared based on a plurality of different standards, respectively;
apply each of a plurality of results of machine learning to all the training images, respectively;
learn by machine learning a diagnosis ambiguous area whose corresponding result of diagnosis is ambiguous based on an area on which different determinations are made between at least two of the results of application;
extract the diagnosis ambiguous area in the pathological specimen image based on a result of the machine learning performed by the diagnosis ambiguous area learning unit; and
generate a diagnosis image that enables the diagnosis ambiguous area that is extracted by the diagnosis ambiguous area extractor to be distinguished from other areas.

2. The image processing apparatus according to claim 1, wherein the processor is configured to learn by machine learning the diagnosis ambiguous area based on the training image in which the diagnosis ambiguous area is marked in advance.

3. The image processing apparatus according to claim 1, wherein the processor is configured to:
perform machine learning on the training image that is created based on a single standard for multiple times;
apply each of a plurality of results of machine learning to the training image; and
learn by machine learning the diagnosis ambiguous area based on an area on which different determinations are made between at least two of results of application.

4. The image processing apparatus according to claim 1, wherein the processor is configured to:
perform machine learning independently on a plurality of different sub training images obtained by randomly thinning data out of the training image that is prepared based on a single standard;
apply each of a plurality of results of machine learning to the training image; and
learn by machine learning the diagnosis ambiguous area based on an area on which different determinations are made between at least two of results of application.

5. The image processing apparatus according to claim 1, wherein the processor is further configured to calculate an analysis adequacy of the pathological specimen image based on the extracted diagnosis ambiguous area,
wherein the processor is configured to generate the diagnosis image that enables the diagnosis ambiguous area that is extracted by the diagnosis ambiguous area extractor to be distinguished from other areas and that contains an analysis adequacy image corresponding to the analysis adequacy.

6. The image processing apparatus according to claim 5, wherein the processor is further configured to set a diagnosis subject area to be diagnosed in the pathological specimen image,
wherein the processor is configured to calculate the analysis adequacy of the diagnosis subject area.

7. The image processing apparatus according to claim 6, wherein the processor is configured to set each of a plurality of areas into which the pathological specimen image is divided as the diagnosis subject area.

8. The image processing apparatus according to claim 7, wherein the processor is configured to generate the diagnosis image that enables the diagnosis ambiguous area that is extracted by the diagnosis ambiguous area extractor to be distinguished from other areas and that contains the analysis adequacy image that allows the diagnosis subject area whose corresponding analysis adequacy is above a reference value from among the diagnosis subject areas to be distinguished from other areas.

9. The image processing apparatus according to claim 6, further comprising:
a display configured to display the pathological specimen image and the diagnosis image; and
an input device configured to receive an operation of specifying a diagnosis subject area in the pathological specimen image,
wherein the processor is configured to set part of the area of the pathological specimen image corresponding to the operation as the diagnosis subject area.

10. An imaging system comprising:
an imaging device including
an illuminator configured to apply illumination light to a pathological specimen,
an imager configured to image light via the pathological specimen, and
an optical system configured to form an image of the light via the pathological specimen on the imaging unit; and
the image processing apparatus according to claim 1 configured to process a pathological specimen image that is captured by the imaging device.

11. A method of processing a pathological specimen image obtained by imaging a pathological specimen, the method comprising:
performing machine learning independently on a plurality of training images for machine learning that are prepared based on a plurality of different standards, respectively;
applying each of a plurality of results of machine learning to all the training images, respectively;
learning by machine learning a diagnosis ambiguous area whose corresponding result of diagnosis is ambiguous based on an area on which different determinations are made between at least two of the results of application;
extracting the diagnosis ambiguous area in the pathological specimen image based on a result of the machine learning performed by the diagnosis ambiguous area learning unit; and
generating a diagnosis image that enables the diagnosis ambiguous area that is extracted by the diagnosis ambiguous area extractor to be distinguished from other areas.

12. A non-transitory computer readable recording medium on which an executable program for processing a pathological specimen image obtained by imaging a pathological specimen is recorded, the program instructing a processor to execute:
performing machine learning independently on a plurality of training images for machine learning that are prepared based on a plurality of different standards, respectively;
applying each of a plurality of results of machine learning to all the training images, respectively;
learning by machine learning a diagnosis ambiguous area whose corresponding result of diagnosis is ambiguous based on an area on which different determinations are made between at least two of the results of application;
extracting the diagnosis ambiguous area in the pathological specimen image based on a result of the machine learning performed by the diagnosis ambiguous area learning unit; and
generating a diagnosis image that enables the diagnosis ambiguous area that is extracted by the diagnosis ambiguous area extractor to be distinguished from other areas.
